(19) United States
(12) Patent Application Publication
(10) Pub. No.: US 2024/0045844 A1
(43) Pub. Date: Feb. 8, 2024

(54) SIGNAL

(71) Applicant: The Johns Hopkins University, Baltimore, MD (US)

(72) Inventors: Christopher Douville, Baltimore, MD (US); Haley Grant, Baltimore, MD (US); Albert Kuo, Baltimore, MD (US); Kamel Lahouel, Baltimore, MD (US); Kenneth W. Kinzler, Frankford, DE (US); Nickolas Papadopoulos, Towson, MD (US); Cristian Tomasetti, Baltimore, MD (US); Bert Vogelstein, Baltimore, MD (US)

(21) Appl. No.: 18/265,118

(22) PCT Filed: Oct. 1, 2021
(86) PCT No.: PCT/US2021/053140

§ 371 Date: Jun. 2, 2023

Related U.S. Application Data
(60) Provisional application No. 63/125,171, filed on Dec. 14, 2020.

(51) Int. Cl. G06F 16/22; G16H 50/70; G16H 50/30

(52) U.S. Cl. CPC G06F 16/22; G16H 50/70; G16H 50/30

(57) ABSTRACT

A method for classifying data using non-negative matrix factorization can include receiving a population of sample data, generating a first matrix of the amplicon counts per sample data, dividing the first matrix into a product of a second matrix and a third matrix, in the second matrix, determining whether each signature is a long or short fragment per each amplicon count, in the third matrix, determining intensities of each signature per the sample data, and classifying the sample data based on the intensities of each signature. The population can include amplicon counts per sample data. The second matrix can include signatures of short and long DNA fragments and the third matrix can include intensities of each signature of the short and long DNA fragments.

### CROSS-REFERENCE TO RELATED APPLICATIONS

This application claims the benefit of U.S. Patent Application No. 63/125,171, filed on Dec. 14, 2020. The disclosure of the prior application is incorporated by reference in its entirety.

### TECHNICAL FIELD

This document describes devices, systems, and methods related to classifying data. In particular, this document relates to classifying amplicon-based sequencing data for early cancer detection and detection of pre-cancer lesions.

### BACKGROUND

Early detection of cancer in a sample or patient can benefit cancer research and treatment.

### SUMMARY

This document generally relates to classifying amplicon-based sequencing data to identify cancer samples from normal samples. Signatures can be generated of DNA fragment lengths to determine a cancer classification. The disclosed techniques can also be applied to detecting adenomatous polyps and/or advanced adenomas in intestine and/or other pre-cancer tumors. In other words, the disclosed techniques can be used not only in cancer classification(s) but also for detection of pre-cancer lesions (e.g., polyps, nodules) and for monitoring and/or early detection of cancer recurrence after surgery.

Although the disclosed inventive concepts include those defined in the attached claims, it should be understood that the inventive concepts can also be defined in accordance with the following embodiments.

Embodiment 1 is a method for classifying data using non-negative matrix factorization, the method comprising receiving a population of sample data, wherein the population includes amplicon counts per sample data, generating a first matrix of the amplicon counts per sample data, dividing the first matrix into a product of a second matrix and a third matrix, the second matrix being signatures of short and long DNA fragments and the third matrix being intensities of each signature of the short and long DNA fragments, in the second matrix, determining whether each signature is a long or short fragment per each amplicon count, in the third matrix, determining intensities of each signature per the sample data, and classifying the sample data based on the intensities of each signature.

Embodiment 2 is the method of embodiment 1, further comprising normalizing the amplicon counts.

Embodiment 3 is the method of any one of embodiments 1 through 2, further comprising filtering the amplicon counts.

Embodiment 4 is the method of any one of embodiments 1 through 3, wherein the signatures include a first signature indicative of the short fragment size and a second signature indicative of the long fragment size.

Embodiment 5 is the method of any one of embodiments 1 through 4, wherein the short fragment size is indicative of cancer.

Embodiment 6 is the method of any one of embodiments 1 through 5, wherein the long fragment size is indicative of normal.

Embodiment 7 is the method of any one of embodiments 1 through 6, further comprising assigning a classifier value of 1 to sample data having a greater intensity of the first signature.

Embodiment 8 is the method of any one of embodiments 1 through 7, further comprising assigning a classifier value of 0 to sample data having a greater intensity of the second signature.

Embodiment 9 is the method of any one of embodiments 1 through 8, further comprising applying a non-negative least square function to the intensities of each signature per each sample data.

Embodiment 10 is the method of any one of embodiments 1 through 9, further comprising applying linear regression analysis to the intensities of each signature per each sample data.

Embodiment 11 is the method of any one of embodiments 1 through 10, wherein classifying the sample data comprises applying a deep learning model.

Embodiment 12 is the method of any one of embodiments 1 through 11, wherein classifying the sample data comprises applying a state vector machine.

Embodiment 13 is the method of any one of embodiments 1 through 12, wherein each sample data is a chromosomal arm.

Embodiment 14 is the method of any one of embodiments 1 through 13, wherein each sample data is a sequenced DNA sample.

Embodiment 15 is the method of any one of embodiments 1 through 14, further comprising iteratively improving one or more algorithms applied in the method.

Embodiment 16 is the method of any one of embodiments 1 through 15, wherein the short fragment size is indicative of at least one of adenomatous polyps or advanced adenomas in an organ or tumor.

Embodiment 17 is a system comprising one or more computers and one or more processors and computer memory storing instructions that, when executed by the processors, cause the processors to perform the method of any one of claims 1 to 16.

The devices, system, and techniques described herein may provide one or more of the following advantages. For example, the disclosed embodiments can assist in testing for cancer and early detection of cancer in a sample or population of samples. Such detection can also be advantageous to improve cancer research across different samples and populations of samples.

As another example, the disclosed embodiments can provide for interpretable results. Lab technicians or experts can receive an easily readable and understandable value that indicates a classification of normal or cancer per sample or patient in a population. For example, a sample that has been classified as cancer can receive a binary value of 1 while a sample that has been classified as normal can receive a binary value of 0. These binary values can be more easily read and interpreted by the lab technicians or experts. Therefore, the lab technicians or experts can more effectively and quickly address samples that have been classified as cancer.

As yet another example, the disclosed embodiments can provide for more accurate performance than existing methodologies for detecting cancer status. Continuous training of algorithms and models used to detect cancer can provide for more accurate and faster cancer classification in subsequent trials. As a result, cancer can be detected earlier and therefore addressed sooner in a sample or patient.

The details of one or more implementations are set forth in the accompanying drawings and the description below. Other features and advantages will be apparent from the description and drawings, and from the claims.

### BRIEF DESCRIPTION OF THE DRAWINGS

FIG. 1 is a conceptual diagram of a system for classifying sequencing data.

FIG. 2 is a flowchart of a process for classifying cancer status in sequencing data.

FIG. 3 is a diagram of system components of the system of FIG. 1.

FIG. 4 is a flowchart of a process for classifying sequencing data.

FIGS. 5A-E depict non-negative matrix factorization of the process of FIG. 4.

FIG. 6 is graphical depictions of classified sequencing data using the techniques described herein.

FIG. 7 is a flowchart of a process for non-negative matrix factorization of FIG. 4.

FIG. 8 depicts an alternative process for filtering training data with lasso logistic regression.

FIG. 9 depicts an alternative process for training a classifier using filtered training data with elastic net regression.

FIG. 10 is a graphical depiction of results from an example blinded case-control study using the disclosed techniques.

FIG. 11 is a graphical depiction of applying the disclosed techniques to replicate samples.

FIG. 12 is a schematic diagram that shows an example of a computing device and a mobile computing device.

Like reference symbols in the various drawings indicate like elements.

### DETAILED DESCRIPTION OF ILLUSTRATIVE EMBODIMENTS

This document generally relates to classifying amplicon-based sequencing data to identify cancer samples from normal samples. Amplicon-based sequencing data can be normalized, filtered, and classified to determine cancer status. For example, amplicons in a chromosomal arm or other DNA sample can be excluded based on size and other factors. The amplicons can then be filtered (e.g., for predictive algorithms using lass logistic regression). Once filtered, signatures can be determined for short and long fragments of each chromosomal arm. An intensity of these signatures can be determined. Chromosomal arms having high intensity of short fragments can be indicative of cancer while chromosomal arms having high intensity of long fragments can be indicative of a normal status (e.g., no cancer). The cancer or normal status classifications can be outputted to a device for viewing and/or use by a lab expert, technician, or other type of speciality. The disclosed techniques can also be applied to detecting adenomatous polyps and/or advanced adenomas in intestine and/or other pre-cancer tumors. In other words, the disclosed techniques can be used not only in cancer classification(s) but also for detection of pre-cancer lesions (e.g., polyps, nodules) and for monitoring and/or early detection of cancer recurrence after surgery.

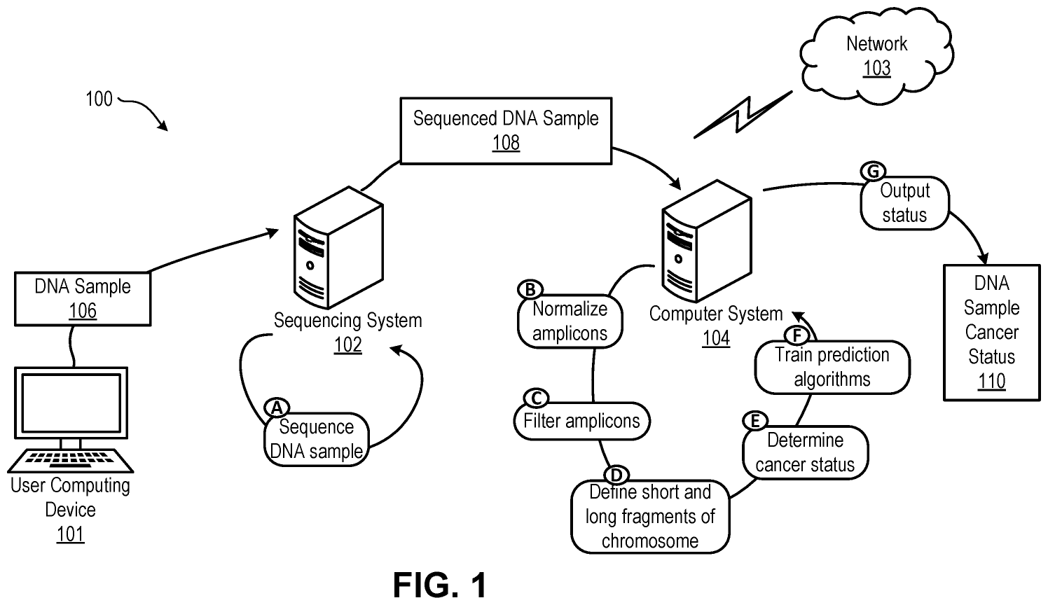

Referring to the figures, FIG. 1 is a conceptual diagram of a system 100 for classifying sequencing data. A user computing device 101, a sequencing system 102, and a computer system 104 can be in communication (e.g., wired, wireless) via a network 103. A lab technician, expert, or other specialist can use the user computing device 101. The lab technician can read a DNA sample 106 into the user computing device 101. The DNA sample 106 can be communicated or transmitted to the sequencing system 102. The sequencing system 102 can sequence the DNA sample (A). Sequenced DNA sample 108 can then be transmitted to the computer system 104. The sequenced DNA sample 108 can be one chromosomal arm for one patient or sample. In other implementations, the sequencing system 102 can transmit a population of sequenced DNA samples to the computer system 104 (e.g., one chromosomal arm per patient or sample in a population).

The computer system 104 can be configured to classify the sequenced DNA sample 108. Classifying the sequenced DNA sample 108 can include identifying a cancer status for that sample 108. The computer system 104 can normalize amplicons of the sample 108 (B). The computer system 104 can also filter the amplicons of the sample 108 (C). Normalizing and filtering the amplicons can be performed in any order and/or simultaneously. In some implementations, the computer system 104 can normalize or filter the amplicons, rather than normalize and filter the amplicons.

Once the amplicons for the sample 108 are normalized and/or filtered (B, C), the computer system 104 can define signatures for short and long fragments of the sample 108 (e.g., of each chromosomal arm) (D). Based on an intensity of the signatures for short or long fragments of the sample 108, the computer system 104 can determine a cancer stats for the sample 108 (E). For example, as described herein, a greater intensity of shorter fragments can indicate cancer associated with that sample 108. On the other hand, a greater intensity of longer fragments can indicate that the sample 108 is normal (e.g., no cancer status).

The computer system 104 can train its prediction algorithms (F). For example, normalizing and filtering algorithms or techniques can be iteratively improved (B, C). Algorithms or techniques used to define short and long fragments (D) can be iteratively improved such that in future classifications, the computer system 104 more accurately identifies short and long fragments. Moreover, algorithms or techniques used to determine cancer status based on intensity of the short and long fragments (E) can be iteratively improved based on historic classifications in order to provide for more accurate cancer status determinations in future classifications.

The determined cancer status(es) can be outputted (G) as DNA sample cancer status 110. For example, the computer system 104 can transmit the DNA sample cancer status 110 to the user computing device 101. The user computing device 101 can then display the status 110 to the lab technician.

In some implementations, the user computing device 101, the sequencing system 102, and/or the computer system 104 can be one central computing system. In other implementations, one or more of the user computing device 101, the sequencing system 102, and/or the computer system 104 can be separate computing system in communication via the network 103.

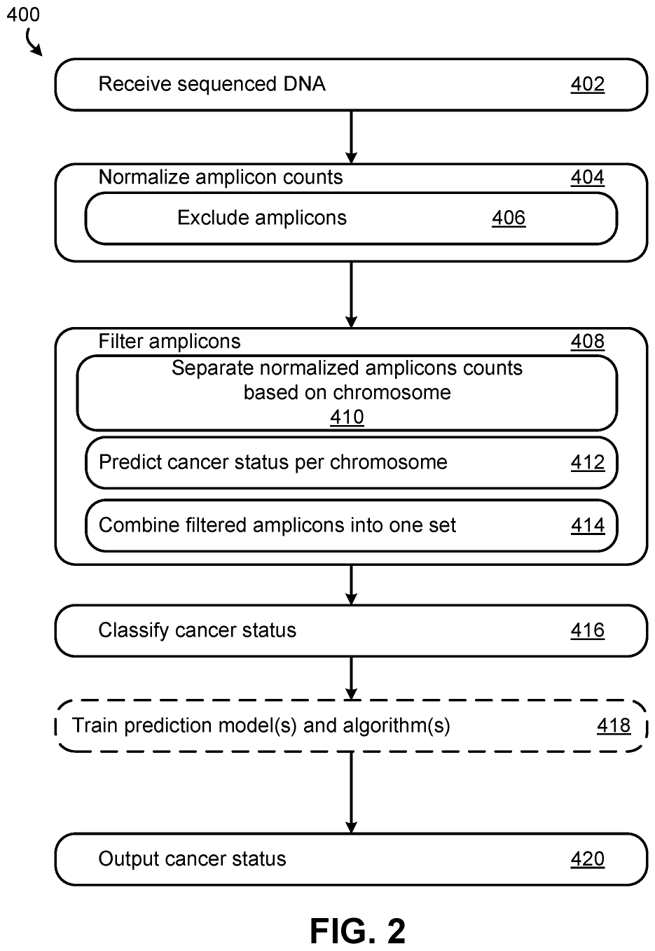

FIG. 2 is a flowchart of a process 400 for classifying cancer status in sequencing data. The process 400 can be performed by the computer system (e.g., refer to the computer system 104 in FIG. 1) and/or any other computer system described herein.

Sequenced DNA can be received in 402. As described throughout, this sequenced DNA can include one chromosomal arm per patient or sample in a population. Amplicon counts for each of the chromosomal arms can be normalized in 404. For example, amplicons can be excluded in 406, as described above.

The amplicons can be filtered in 408. For example, the normalized amplicon counts can be separated based on chromosome in 410. A cancer status can be predicted per chromosome in 412. Moreover, these filtered amplicons can be combined into one set in 414.

Normalizing amplicons in 404 and/or filtering amplicons in 408 can include using the normal samples in a training set to perform a 3 way ANOVA, where factors are primer lots, cohorts, and races. A p-value associated with each individual factor can be identified and used to exclude any amplicons having a corresponding p-value less than 0.01 in any of these 3 factors. Additionally or alternatively, amplicons can be excluded where a correlation between a (non-normalized) count (e.g., number of reads for that amplicon) and a total number of reads across all amplicons in the corresponding chromosome is less than 0.8. Additionally or alternatively, the computer system can keep only amplicons having a length greater than or equal to 81 and having a mean normalized count larger in normals than in cancers, and amplicons having a length less than or equal to 81 and having a mean normalized count larger in cancers than in normals.

In 416, cancer status can be classified. Classifying cancer status can be performed for each chromosome as well as for each chromosomal arm. A fundamental metric on which classification is based on is the normalized amplicon counts, defined by the number of reads for amplicon i divided by the total number of reads across all amplicons in the corresponding chromosome arm. Alternatively, it is possible to use the total number of reads across all amplicons of one chromosome or of all chromosomes.

Classifying the cancer status in 416 can also include applying one or more classifiers, such as a logistic regression or a Gaussian kernel SVM.

Once cancer status is determined in 416, the computer system can optionally train prediction model(s) and/or algorithm(s) in 418. Training such models and/or algorithms can be beneficial to improve accuracy of the computer system in normalizing, filtering, and classifying cancer status, as described herein.

The determined cancer status per chromosome and/or per chromosomal arm can be outputted in 420. Outputting the cancer status can be advantageous to provide a lab technician with interpretable results.

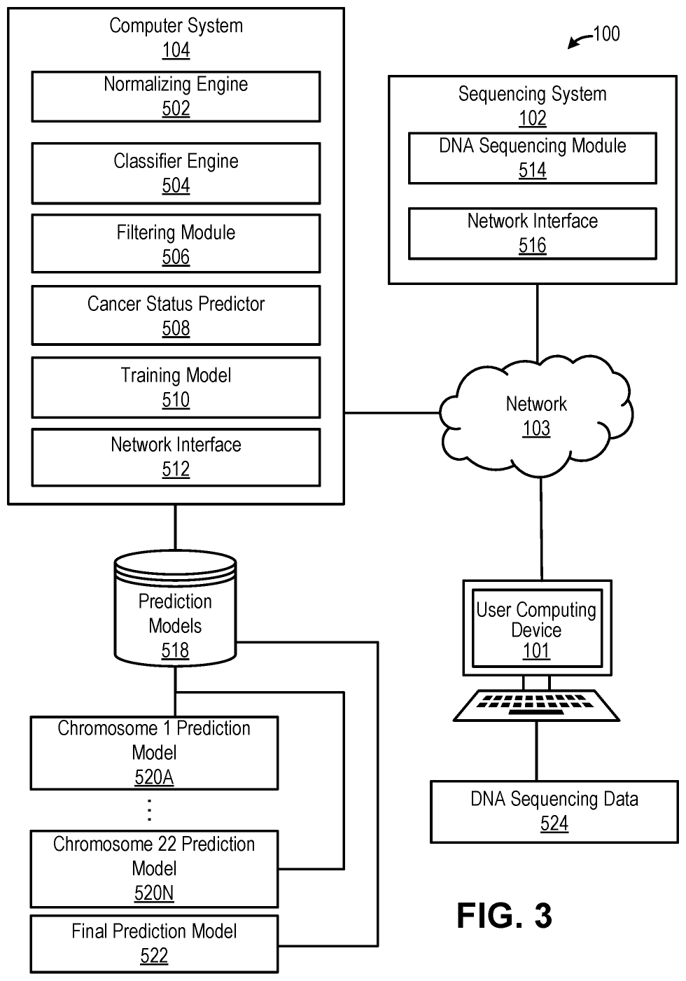

FIG. 3 is a diagram of system components of the system 100 of FIG. 1. As described above, the system 100 includes the user computing device 101, the sequencing system 102, and the computer system 104, which can communicate via the network 103.

The user computing device 101 can provide a user such as a lab technician with a display, input, and output devices. The user can provide DNA sequencing data 524 to the user computing device 101, which can transmit that data 524 to the sequencing system 102 and/or the computer system 104.

The sequencing system 102 can include a DNA sequencing module 514 and a network interface 516. One or more processors of the sequencing system 102 can be configured to perform operations such as sequencing the data 524 in the module 514. The network interface 516 can provide for communication between one or more components of the system 100.

The computer system 104 can include a normalizing engine 502, a classifier engine 504, a filtering module 506, a cancer status predictor 508, a training model 510, and a network interface 512. One or more of these components of the computer system 104 can be combined and/or removed from the system 104.

The normalizing engine 502 can be configured to normalize data. For example, the computer system 104 can receive sequenced DNA data from the sequencing system 102. The normalizing engine 502 can then normalize (e.g., exclude) amplicons of the sequenced DNA data.

The filtering module 506 can be configured to filter the normalized amplicons, as described herein. The normalizing engine 502 and the filtering module 506 can be a same engine in some implementations.

The cancer status predictor 508 can be configured to perform non-negative matrix factorization, as described herein. The predictor 508 can generate matrixes, identify signatures for short and long fragments, and determine signature intensities per DNA sample.

The classifier engine 504 can then classify each DNA sample as cancer or normal based on analysis of the signature intensities. The classifier engine 504 can be an SVM and/or a LASSO regression, as described herein.

The training model 510 can be configured to train and/or improve algorithms and/or models that are used by the system 104 in normalizing, filtering, predicting cancer status, and classifying. As a result, the algorithms and/or models implemented by the computer system 104 can be continuously improved such that the computer system 104 can more accurately predict cancer status in future classifications.

The network interface 512 can provide for communication between the computer system 104 and one or more other components of the system 100.

The computer system 104 can be in communication with a prediction models database 518. The database 518 can be configured to store prediction models for chromosomes 1 through 22 520A-N as well as a final prediction model 522. For example, the chromosome prediction models 520A-N can be used in classifying or identifying cancer status in each individual chromosome. The final prediction model 522 can be used to identify an overall cancer status for a particular sample. As described herein, the cancer status predictor 508 can be configured to use the chromosome prediction models 520A-N and the classifier engine 504 can be configured to use the final prediction model 522, which can be based on cancer status per chromosome as determined by the cancer status predictor 508. Moreover, the models 520A-N and 522 can be updated and/or modified over time by the training model 510. These models 520A-N and 522 can be improved such that they more accurately predict cancer status in chromosomes and samples.

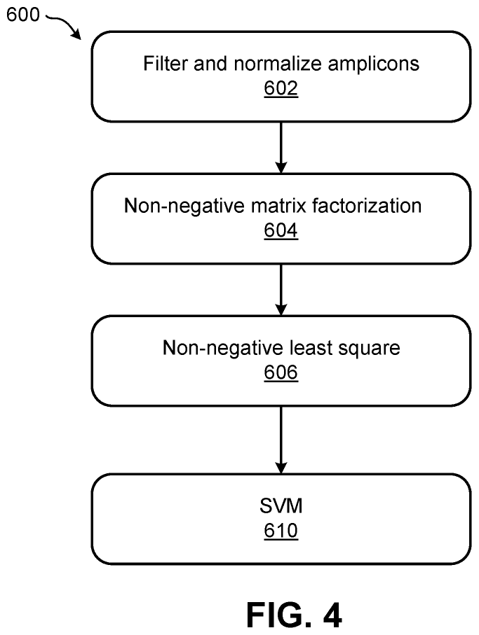

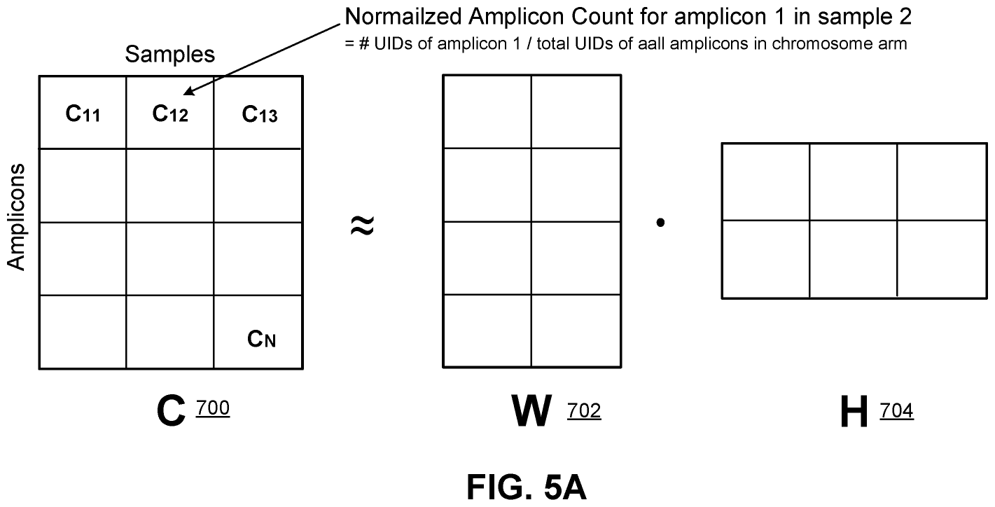

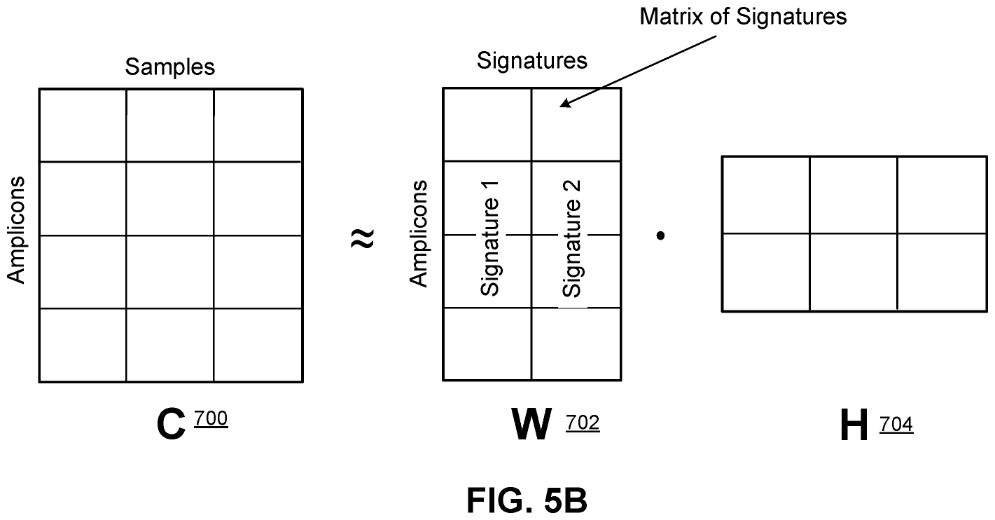

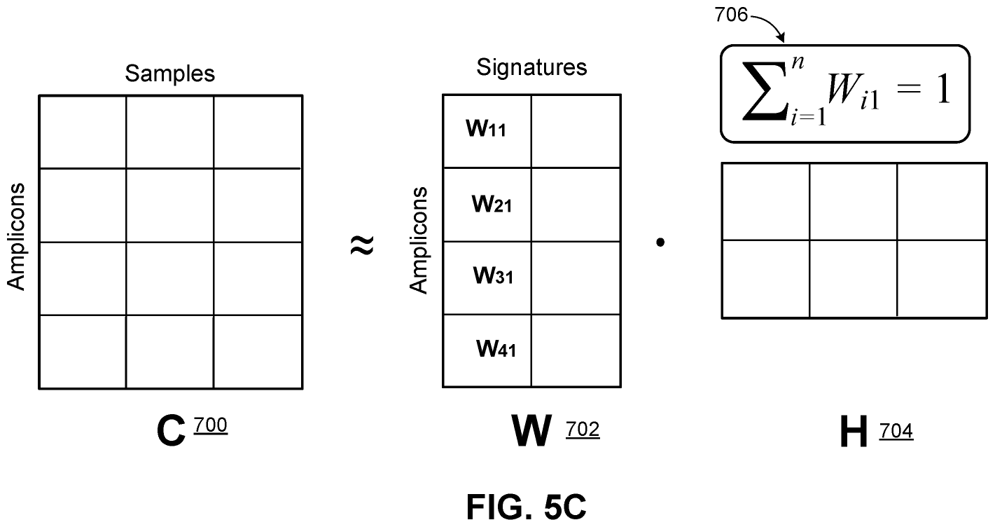

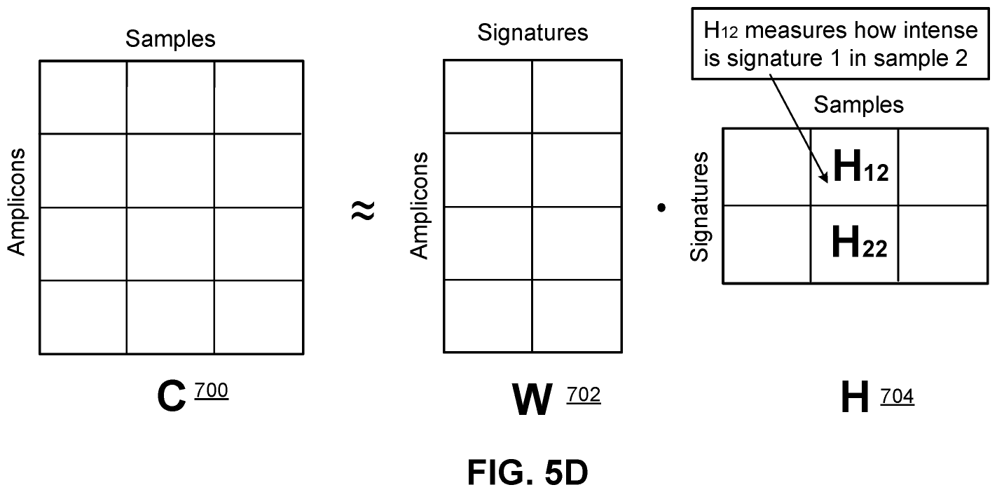

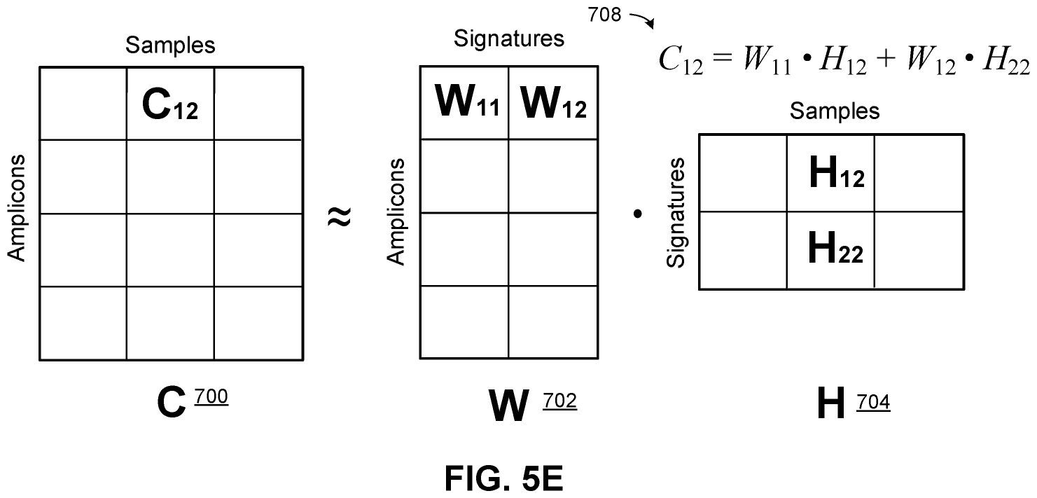

FIG. 4 is a flowchart of a process 600 for classifying sequencing data. The process 600 can be performed by the computer system, as described herein. FIGS. 5A-E depict non-negative matrix factorization of the process 600 of FIG. 4. Referring to FIGS. 4-5, amplicons from DNA samples can be filtered and normalized in 602.

Non-negative matrix factorization can be performed in 604 (e.g., refer to FIGS. 5A-E). For example, one chromosome can be fixed. M^{TrainNormal} can be defined as a matrix of normal training where every column can be one individual and every row can be one amplicon. An entry M^{TrainNormal}ᵢⱼ can therefore the normalized count of amplicon i in individual j. In the same way other matrixes, such as M^{TrainCancer}, M^{TestNormal} and M^{TestCancer}. Finally, M^{Train} can be defined as a matrix (e.g., matrix 700 in FIGS. 5A-E) where all training data regardless of class can be concatenated:

M^{Train}= [symbol] (M^{TrainNormal}, M^{TrainCancer}).

Non-negative matrix factorization (NMF) decomposition can then be computed for M (e.g., refer to matrixes 702 and 704 in FIGS. 5A-E):

M^{Train}˜W^{Train}H^{Train}

It can be assumed that each column of W^{Train} (e.g., the matrix 702 in FIGS. 5A-E) sums to 1. Every column in W^{Train} can define a distribution on the amplicons and can be associated to one factor (e.g., signature, feature) as follows. The distribution can yield a distribution over lengths to which a mean length can be associated. Using these means, short factors, long factors, and neutral factors can be defined. The short factors are factors where the associated mean length can be less than a ⅓ quantile of the means. The long factors are the factors where the associated mean length can be larger than a ⅔ quantile of the means. The neutral factors can be any remaining factors.

The factors are signatures for short and long fragments. Each row of H^{Train} (e.g., the matrix 704 in FIGS. 5A-E) can also be associated to one factor. The factors are signatures for be short and long fragments.

W^{Train} can be stored and/or fixed while each column of H^{Train} can be recomputed (e.g., each column of H^{Train} corresponds to one individual/patient/sample and represents a features vector of that individual/patient/sample).

To compute the features matrix of a test set H^{Test}, a non-negative least squares (NNLS) regression in 606 can be performed:

M^{Test}[,j]˜W^{Train}H^{Test}[,j].

The intensities of all factors (e.g., signatures) obtained with NNLS for each sample, combined with a cancer status of that sample, can then be used to train and classify samples as normal or cancer, by training a classifier, such as support vector machines (SVM) or logistic regression.

SVM can be used as supervised learning models having associated learning algorithms. Thus, SVMs can be beneficial to analyze data, such as the DNA samples, to more accurately classify that data to be indicative of cancer or normal. A Gaussian kernel SVM can use all factors as features without any constraint. As another example, a Gaussian kernel SVM can be used with the following additional constraint: the computer system can keep only short factors where a median among normals is lower than a median among cancers. The additional constraint can also require the computer system to keep only long factors where the median among normals is higher than the median among cancers. All neutral factors can also be kept.

Logistic regression can additionally or alternatively be used in 610 to classify the DNA samples as normal or cancer. In logistic regression, a coefficient associated with long fragments (e.g., factors) can be negative. A coefficient associated with short fragments can be positive. A coefficient associated with neutral fragments can be without sign constraints.

In an example where only short and long factors are defined, there are no neutral factors. The short factors can be factors where the associated mean length is less than the median of mean lengths associated to the factors. The long factors can be the factors where the associated mean length is larger than the median of the means. Then, a logistic regression classifier can be used where a coefficient associated to the long factors is negative and the coefficient associated to the short factors is positive. An additional or alternative classifier can be a Gaussian kernel SVM using all factors (short and long only) as features without any constraint. An additional or alternative classifier can be a Gaussian kernel SVM in which only short factors are kept where the median among normals is lower than the median among cancers and/or only long factors are kept were the median among normals is higher than the median among cancers.

Moreover, in some implementations, to get more stable classifications (normal versus cancer), the training set of data can be split into two parts. A first part can be used to compute a W^{Train} matrix, which can be denoted as W^{Train}₁. Then, a non-negative least squares regression can be applied to W^{Train} in order to compute a matrix H^{Train} on the entire training set. H^{Test} can then be computed using W^{Train}₁. Now that features are identified, the computer system can apply a classification method (e.g., the SVM in 610) to obtain a first score. This process can be repeated using a second part of the training set of data and computing a matrix W^{Train}₂. A second score can be generated. The two scores can be combined using a Fisher method.

Moreover, in some implementations, an additional filtering of the amplicons can be performed. For every chromosome, the computer system can take the normalized counts of amplicons and feed them to a logistic LASSO classifier with constraints that the coefficients of the lasso are negative for amplicons of size>81 and positive for amplicons of size<81. As described throughout, shorter or smaller sized amplicons are indicative of cancer. A sign of the coefficients of amplicons of size=81 can be kept free (e.g., these are neutral factors, fragments, or features). The amplicons selected by the LASSO model can be ones that are kept for the steps discussed below. Next, for every chromosome, the filtered set of amplicons can be used to estimate a probability: P(Reading fragment|length of fragment=L) . Moreover, a quantity that is proportional to the former probability can be estimated. The former probability can be proportional to:

$$\frac{P ( length of fragment = L \vert Reading fragment )}{P ( length of fragment = L )}$$

The probability P(Reading fragment|length of fragment=L) can be estimated by a proportion of amplicons having length L. The probability P(length of fragment=L|Reading fragment) can be estimated by a sum of normalized reads of filtered amplicons having length L.

Finally, using all estimated probabilities P(Reading fragment|length of fragment=L) for all possible lengths and all chromosomes and feeding them to an elastic net classifier, the coefficient can be imposed as positive when L<81 (e.g., indicative of cancer) and negative when L>81 (e.g., indicative of normal).

FIGS. 5A-E depict non-negative matrix factorization of the process 600 of FIG. 4. As described above in reference to FIG. 4 and depicted in FIG. 5A, the matrix 700 can represent a population of samples. A standard distribution of different amplicons can be identified to then determine whether any one of the samples represented in the matrix 700 has a higher number or intensity of longer fragments or shorter fragments. Each sample, such as C₁₁, C₁₂, C₁₃, and C_{N} in the matrix 700 can have a normalized amplicon count. The normalized amplicon count can be a number of UIDs of one amplicon divided by a total number of UIDs of all amplicons in one chromosomal arm. The matrix 700 can be broken into a product of two matrixes, 702 and 704. In both matrixes 702 and 704, there may be no negatives.

As depicted in FIG. 5B and described above in reference to FIG. 4, signatures can be generated for short fragments and long fragments. The signatures can be represented in the matrix 702. Signature 1 can represent short fragments. Signature 2 can represent long fragments.

As depicted in FIG. 5C, each signature can have a probability value. Weights can be assigned to each amplicon per signature in the matrix 702. In other words, the signatures can be weighted and/or normalized. Exemplary weights for signature 1 (short fragments) include W₁₁, W₂₁, W₃₁, and W₄₁. The weights of the signature in the matrix 701 can be added up to equal 1, as demonstrated in equation 706.

FIG. 5D demonstrates the matrix 704, which can be used to determine how intense a signature is in a particular sample of the population. A first row in the matrix 704 can represent signature 1 (short fragments) and a second row in the matrix 704 can represent signature 2 (long fragments). If, for example, sample 2 has an intense H₁₂ of signature 1 that can indicate that the patient has short fragments, which is cancer. On the other hand, if sample 2 has an intense H₂₂ of signature 2 that can indicate that the patient has long fragments, which is normal. Relative intensity of short and long fragments per sample can be determined to identify whether the sample has more short fragments or more long fragments. Therefore, higher intensity of signature 1 means that the sample has shorter fragments indicative of cancer. This can provide for a more reliable and accurate classification of cancer status once the intensities of each signature per sample are fed into an SVM or other classifier as mentioned throughout this disclosure.

FIG. 5E demonstrates an equation 708 for determining a classification for the sample C₁₂. As described in reference to FIGS. 5A-D, the classification for the sample can be a weight of the first signature multiplied by the intensity of that first signature plus a weight of the second signature multiplied by the intensity of that second signature. In other words, C₁₂=W₁₁*H₁₂+W₁₂*H₂₂. The resulting numeric value can be used to indicate whether the sample C₁₂ has predominantly short fragments, which is indicative of cancer, or predominantly long fragments, which is indicative of normal.

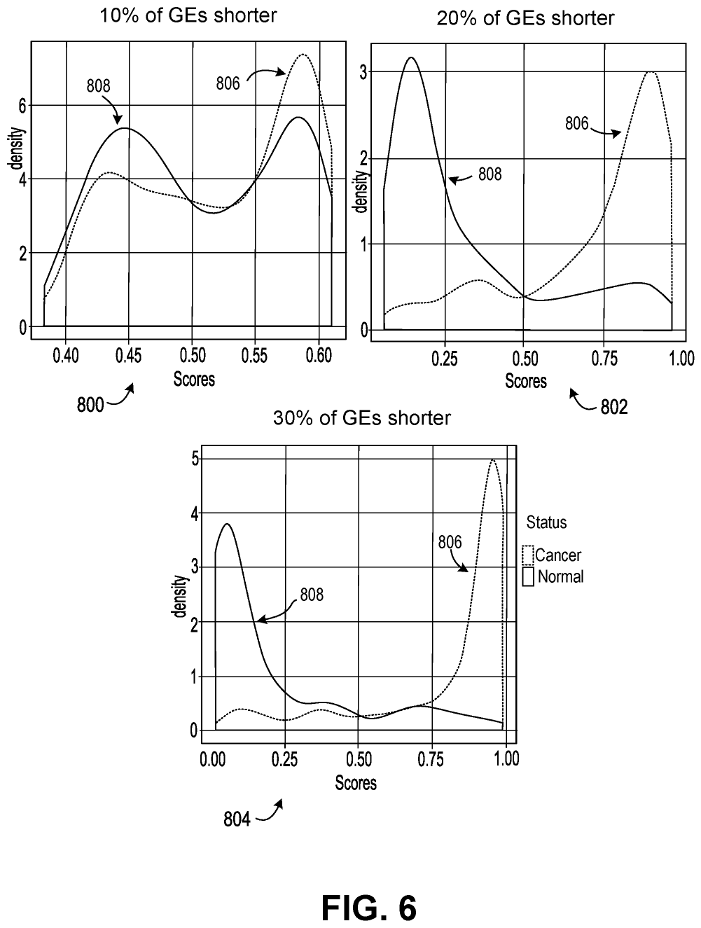

FIG. 6 is graphical depictions of classified sequencing data using the techniques described herein. In graphs 800, 802, and 804, line 806 represents cancer and line 808 represents normal. As depicted in graph 800, when fewer fragments are shorter and only 10% of the genome equivalents have shorter fragments, the cancer line 806 is closer to the normal line 808. As the proportion of more fragmented genome equivalents increases to 20%, the cancer line 806 is more defined and farther away from the normal line 808, as depicted in the graph 802. Finally, in graph 804, when the proportion of more fragmented genome equivalents increases to 30%, the cancer line 806 is clearly more defined and farther away from the normal line 808. Thus, the graphs 800, 802, and 804 indicate a greater accuracy in differentiating, detecting, and identifying cancer when more DNA samples are used.

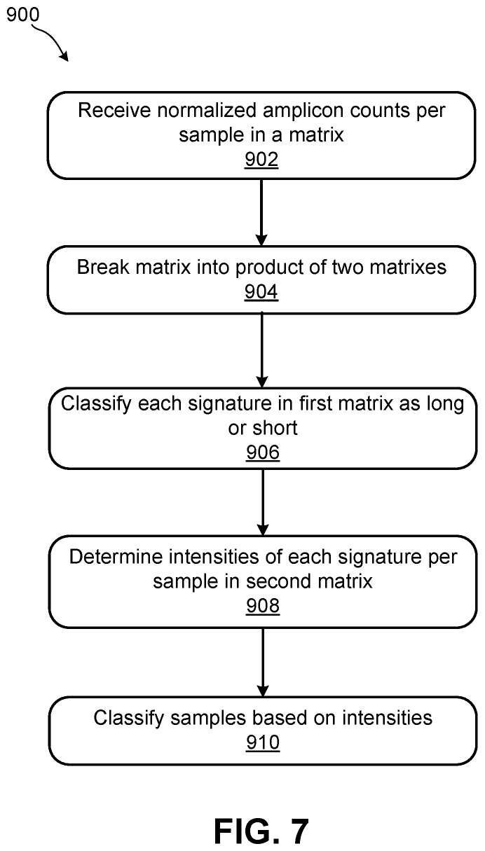

FIG. 7 is a flowchart of a process 900 for non-negative matrix factorization of FIG. 4. As described above in reference to FIGS. 4-5, normalized amplicon counts per sample can be received in a matrix in 902. The matrix can be broken into a product of two matrixes in 904. Each signature can be classified as short or long in the first matrix in 906. Then, an intensity of each signature per sample can be determined in the second matrix in 908. The samples can then be classified as cancer or normal based on the intensities in 910.

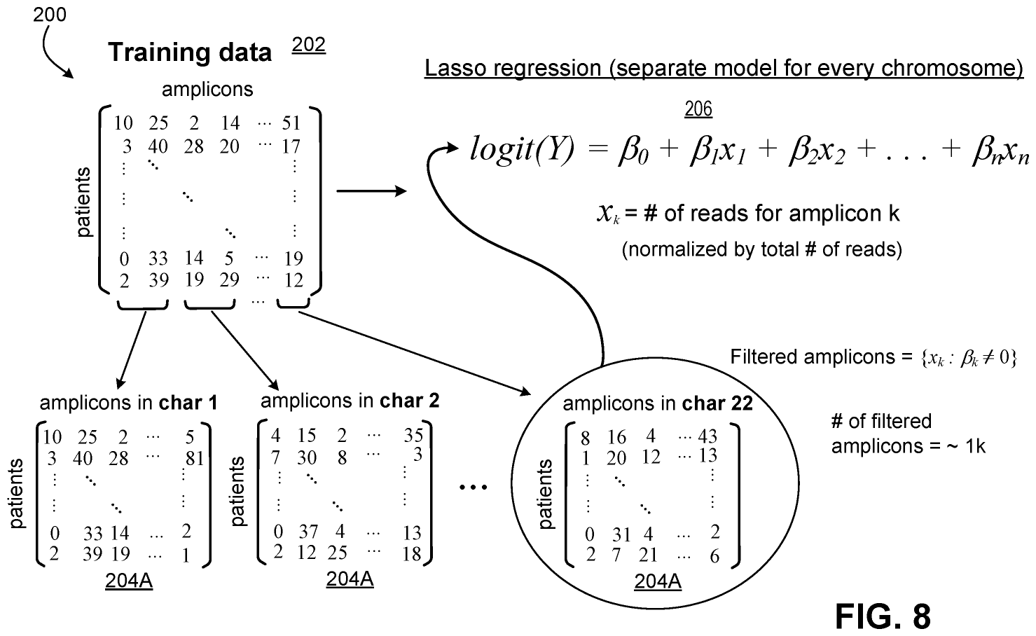

FIG. 8 depicts an alternative process for filtering 200 training data 202 with lasso logistic regression 206. This can be an alternative approach to the systems and methods described herein. The training data 202 can include amplicons per chromosomal arm 204A-N (e.g., the sequenced DNA sample 108) that is received by the computing system 104 (e.g., refer to FIG. 1).

The training data 202 can include amplicons 204A-N that were not excluded based on size and other factors. In other words, the amplicons can be normalized. Amplicons can be excluded from a DNA sample based on flagged positions, ambiguous size (e.g., size=0), size being greater than 110 bp, inadequate representation in every race (e.g., an amplicon should have >+20 reads (UID) in >20% of samples in every race in a set of samples; filtering for how frequently the amplicon is read overall; filtering alternatives based on variance and mean count), and/or amplicons on contigs. One or more other factors can be used for excluding amplicons in the DNA sample.

As an example, the computer system can start with or receive 700,000 amplicons. Amplicons can be excluded based on whether they have ambiguous size and size<110 bp. After this step, the computer system can have 400,000 remaining amplicons. The 400,000 remaining amplicons can further be tailored based on keeping amplicons that are represented in every race. As a result, the computer system can be left with 200,000 amplicons to filter and classify.

As depicted in FIG. 8, the normalized amplicons 204A-N can be filtered for predictive amplicons by running the lasso logistic regression 106 on the normalized amplicon counts 204A-N to predict cancer status in every chromosome. The lasso regression 206 can have a feature selection, enabling the computer system to reduce a set of all amplicons 204A-N. In the example above, the set of all amplicons can include 200,000 amplicons and the logistic regression 206 can reduce that number to approximately 1,000 amplicons.

Specifically, within the training data 202, the computer system can separate the amplicons based on which chromosome they belong to (e.g., refer to the amplicon sets per chromosome 204A-N). Then, using the amplicons' normalized reads from a given chromosome (e.g., 204A-N), the computer system can predict cancer status (e.g., normal versus cancer) per chromosome, as described herein. The reads can be normalized by a total number of reads in each sample. This process can be repeated for each chromosome 1 to 22. The filtered amplicons from each chromosome can be combined into one step.

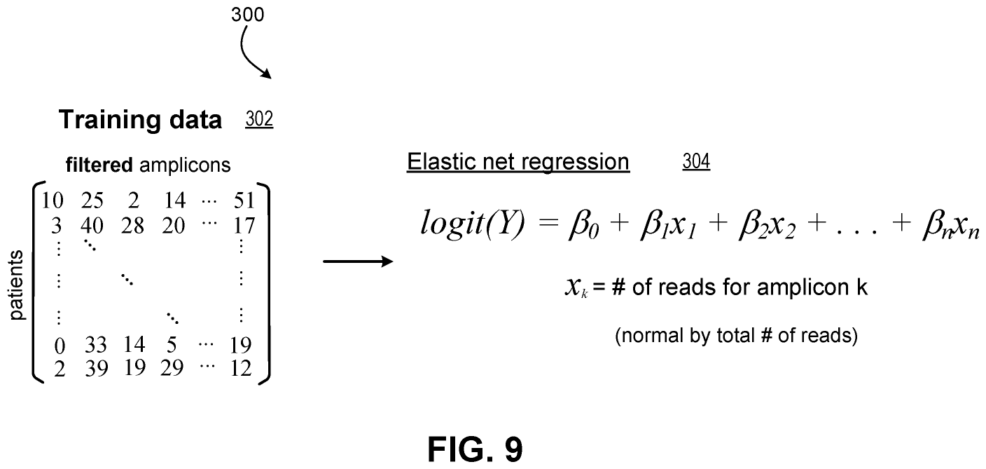

FIG. 9 depicts an alternative process for training 300 a classifier using filtered training data 302 with elastic net regression 304. This can be an alternative approach to the systems and methods described herein. The training 300 can be performed by the computer system described herein. The training 300 can be performed after the amplicons are normalized and/or filtered, as described above (e.g., refer to FIG. 8). For example, the training data 302 can be the data 202 that was filtered as depicted in FIG. 8.

Once the set of filtered amplicons is generated as the training data 302 (e.g., refer to FIG. 8), the computer system can run a final prediction model on the normalized amplicon reads for those filtered amplicons in the training set 302. Among the classifiers, lasso logistic regression, elastic net logistic regression 304, and boosting can be used. Elastic net regression 304 can be more advantageous in terms of speed and performance when classifying the training data 302. In general, a 2-fold cross-validation can be performed with 5 iterations.

Alternatively or in addition, the amplicon count can be normalized by the total number of reads in that amplicon's chromosome instead of the total number of reads overall. Let xₖ be the number of reads for amplicon k in chromosome j. normalizing by the total number of reads can provide a normalized count of

$$\frac{{x}_{k}}{{\sum }_{j = 1}^{22} \sum \text{?}} .$$

$$\text{?} \text{indicates text missing or illegible when filed}$$

In contrast, normalizing by the chromosome total can provide a normalized count for amplicon k in chromosome j of

$$\frac{{x}_{k}}{\sum \text{?}} .$$

$$\text{?} \text{indicates text missing or illegible when filed}$$

Then, in the filtering of amplicons (e.g., refer to FIG. 8), the filtered amplicons can be kept separate by chromosome. The prediction model can be trained for every chromosome on the filtered amplicon read counts, which are now normalized by the chromosome totals. In other words, the computer system can train and test using the filtered amplicons from chromosome 1 only, then the computer system can train and test using the filtered amplicons from chromosome 2 only, and so on. As a result, if the computer system ran 1 final prediction model previously, the computer system can now run 1*22 models, where 22 is the number of chromosomes.

As an example, suppose there is double the number of chromosome j and thus double the number of counts for all the amplicons in chromosome j for cancer patients. Then, dividing by the total number of reads in chromosome j can eliminate this aneuploidy difference between normal and cancer patients. However, dividing by the total number of reads overall can in general not eliminate this aneuploidy signal. This implies that any aneuploidy signal can be reflected in the difference in performance between the two normalization options described herein.

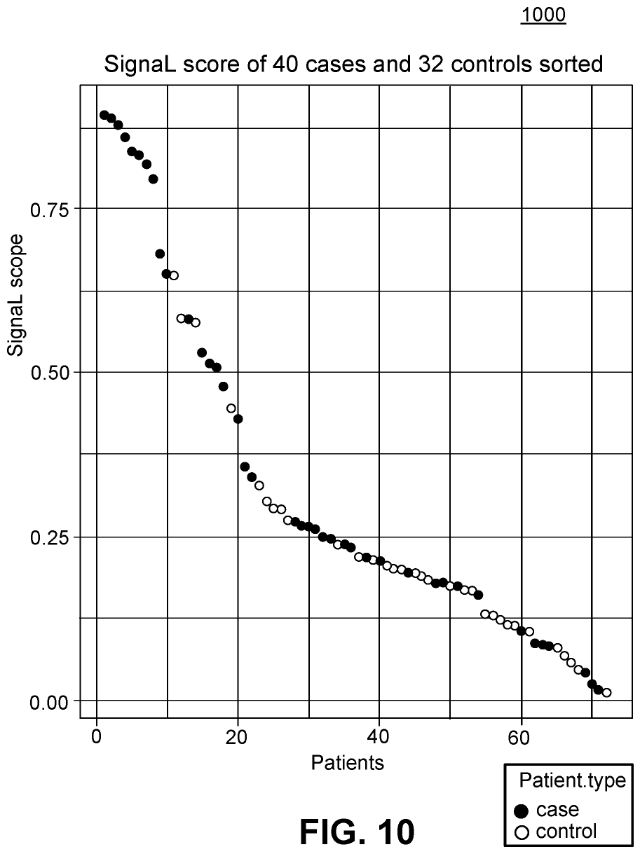

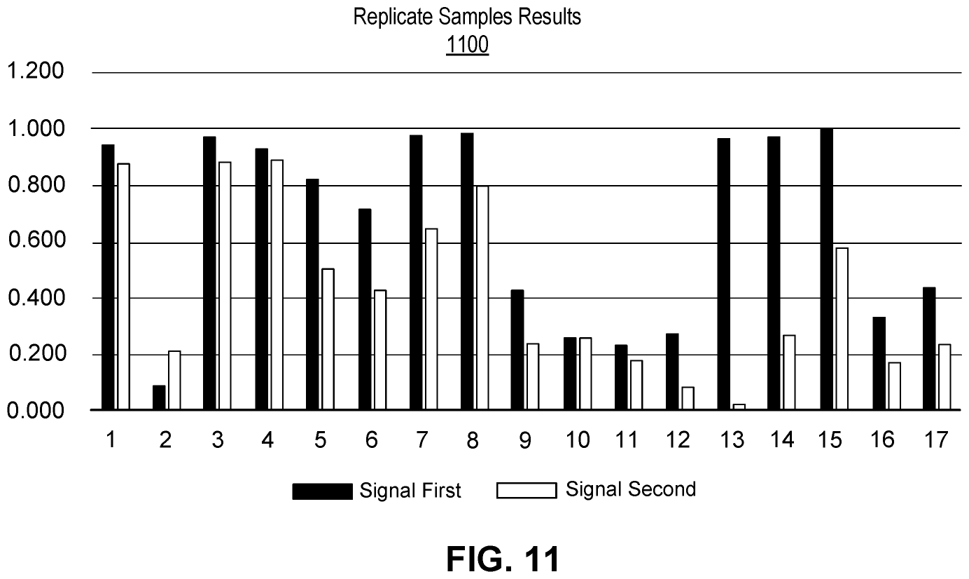

FIG. 10 is a graphical depiction 1000 of results from an example blinded case-control study using the disclosed techniques. FIG. 11 is a graphical depiction 1100 of applying the disclosed techniques to replicate samples. Referring to both FIGS. 10-11, the disclosed techniques can also be used to detect advanced adenoma (AA). For example, the disclosed techniques can provide for detecting, in cfDNA, the presence of aneuploidy and/or an abnormal distribution of DNA fragment length. For example, short DNA fragment size can be indicative of at least one of adenomatous polyps or advanced adenomas in an organ or tumor. After all, a signal provided by aneuploidy or by an abnormal fragment length distribution can be more extensive than one provided by a single mutation. Thus, the disclosed techniques provide for detecting and quantifying presence of “signatures” of aneuploidy and abnormal DNA fragmentation in cfDNA with good sensitivity at high specificity.

As shown by the graphical depiction 1000 of FIG. 10, the disclosed techniques can provide for identification of 8/20 (40%) of AAs, which can be considered an improvement on 8.1% detection rate of AAs using a mutation-based approach.

Both FIGS. 10-11 illustrate an example study, in which 72 blinded blood samples, specifically 40 patients with AA and 32 controls can be tested using the disclosed techniques. The methodology described herein can identify 10/40 (25%) AA at 100% specificity, 11/40 (27.5%) with two false positive (0.94 spec), 15/40 (37.5%) with 3 false positives (0.91 spec), and 19/40 (47.5%) with 4 false positives (0.875 spec) (e.g., refer to FIG. 10). Keeping the same 0.99 specificity threshold that was originally obtained by training the disclosed techniques on cancer data, the performance remains essentially unchanged. FIG. 11 shows a high consistency between original and repeated analyses, thereby demonstrating a high correlation between the first and second score provided using the disclosed techniques. Overall, as shown in FIGS. 10-11, the disclosed techniques can provide for detecting 47.5% of AA, at 87.5% specificity. Importantly, the results of validation using the same threshold obtained in training can highlight reproducibility of the disclosed techniques.

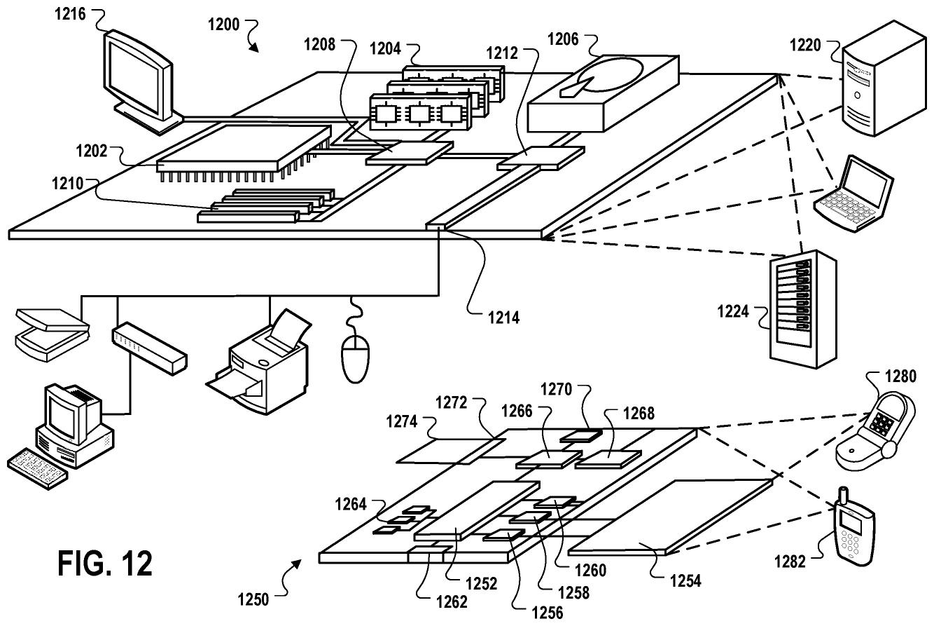

FIG. 12 shows an example of a computing device 1200 and an example of a mobile computing device that can be used to implement the techniques described here. The computing device 1200 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 1200 includes a processor 1202, a memory 1204, a storage device 1206, a high-speed interface 1208 connecting to the memory 1204 and multiple high-speed expansion ports 1210, and a low-speed interface 1212 connecting to a low-speed expansion port 1214 and the storage device 1206. Each of the processor 1202, the memory 1204, the storage device 1206, the high-speed interface 1208, the high-speed expansion ports 1210, and the low-speed interface 1212, are interconnected using various busses, and can be mounted on a common motherboard or in other manners as appropriate. The processor 1202 can process instructions for execution within the computing device 1200, including instructions stored in the memory 1204 or on the storage device 1206 to display graphical information for a GUI on an external input/output device, such as a display 1216 coupled to the high-speed interface 1208. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system).

The memory 1204 stores information within the computing device 1200. In some implementations, the memory 1204 is a volatile memory unit or units. In some implementations, the memory 1204 is a non-volatile memory unit or units. The memory 1204 can also be another form of computer-readable medium, such as a magnetic or optical disk.

The storage device 1206 is capable of providing mass storage for the computing device 1200. In some implementations, the storage device 1206 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer- or machine-readable medium, such as the memory 1204, the storage device 1206, or memory on the processor 1202.

The high-speed interface 1208 manages bandwidth-intensive operations for the computing device 1200, while the low-speed interface 1212 manages lower bandwidth-intensive operations. Such allocation of functions is exemplary only. In some implementations, the high-speed interface 1208 is coupled to the memory 1204, the display 1216 (e.g., through a graphics processor or accelerator), and to the high-speed expansion ports 1210, which can accept various expansion cards (not shown). In the implementation, the low-speed interface 1212 is coupled to the storage device 1206 and the low-speed expansion port 1214. The low-speed expansion port 1214, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter.

The computing device 1200 can be implemented in a number of different forms, as shown in the figure. For example, it can be implemented as a standard server 1220, or multiple times in a group of such servers. In addition, it can be implemented in a personal computer such as a laptop computer 1222. It can also be implemented as part of a rack server system 1224. Alternatively, components from the computing device 1200 can be combined with other components in a mobile device (not shown), such as a mobile computing device 1250. Each of such devices can contain one or more of the computing device 1200 and the mobile computing device 1250, and an entire system can be made up of multiple computing devices communicating with each other.

The mobile computing device 1250 includes a processor 1252, a memory 1264, an input/output device such as a display 1254, a communication interface 1266, and a transceiver 1268, among other components. The mobile computing device 1250 can also be provided with a storage device, such as a micro-drive or other device, to provide additional storage. Each of the processor 1252, the memory 1264, the display 1254, the communication interface 1266, and the transceiver 1268, are interconnected using various buses, and several of the components can be mounted on a common motherboard or in other manners as appropriate.

The processor 1252 can execute instructions within the mobile computing device 1250, including instructions stored in the memory 1264. The processor 1252 can be implemented as a chipset of chips that include separate and multiple analog and digital processors. The processor 1252 can provide, for example, for coordination of the other components of the mobile computing device 1250, such as control of user interfaces, applications run by the mobile computing device 1250, and wireless communication by the mobile computing device 1250.

The processor 1252 can communicate with a user through a control interface 1258 and a display interface 1256 coupled to the display 1254. The display 1254 can be, for example, a TFT (Thin-Film-Transistor Liquid Crystal Display) display or an OLED (Organic Light Emitting Diode) display, or other appropriate display technology. The display interface 1256 can comprise appropriate circuitry for driving the display 1254 to present graphical and other information to a user. The control interface 1258 can receive commands from a user and convert them for submission to the processor 1252. In addition, an external interface 1262 can provide communication with the processor 1252, so as to enable near area communication of the mobile computing device 1250 with other devices. The external interface 1262 can provide, for example, for wired communication in some implementations, or for wireless communication in other implementations, and multiple interfaces can also be used.

The memory 1264 stores information within the mobile computing device 1250. The memory 1264 can be implemented as one or more of a computer-readable medium or media, a volatile memory unit or units, or a non-volatile memory unit or units. An expansion memory 1274 can also be provided and connected to the mobile computing device 1250 through an expansion interface 1272, which can include, for example, a SIMM (Single In Line Memory Module) card interface. The expansion memory 1274 can provide extra storage space for the mobile computing device 1250, or can also store applications or other information for the mobile computing device 1250. Specifically, the expansion memory 1274 can include instructions to carry out or supplement the processes described above, and can include secure information also. Thus, for example, the expansion memory 1274 can be provide as a security module for the mobile computing device 1250, and can be programmed with instructions that permit secure use of the mobile computing device 1250. In addition, secure applications can be provided via the SIMM cards, along with additional information, such as placing identifying information on the SIMM card in a non-hackable manner.

The memory can include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product can be a computer- or machine-readable medium, such as the memory 1264, the expansion memory 1274, or memory on the processor 1252. In some implementations, the computer program product can be received in a propagated signal, for example, over the transceiver 1268 or the external interface 1262.

The mobile computing device 1250 can communicate wirelessly through the communication interface 1266, which can include digital signal processing circuitry where necessary. The communication interface 1266 can provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication can occur, for example, through the transceiver 1268 using a radio-frequency. In addition, short-range communication can occur, such as using a Bluetooth, WiFi, or other such transceiver (not shown). In addition, a GPS (Global Positioning System) receiver module 1270 can provide additional navigation- and location-related wireless data to the mobile computing device 1250, which can be used as appropriate by applications running on the mobile computing device 1250.

The mobile computing device 1250 can also communicate audibly using an audio codec 1260, which can receive spoken information from a user and convert it to usable digital information. The audio codec 1260 can likewise generate audible sound for a user, such as through a speaker, e.g., in a handset of the mobile computing device 1250. Such sound can include sound from voice telephone calls, can include recorded sound (e.g., voice messages, music files, etc.) and can also include sound generated by applications operating on the mobile computing device 1250.

The mobile computing device 1250 can be implemented in a number of different forms, as shown in the figure. For example, it can be implemented as a cellular telephone 1280. It can also be implemented as part of a smart-phone 1282, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of the disclosed technology or of what may be claimed, but rather as descriptions of features that may be specific to particular embodiments of particular disclosed technologies. Certain features that are described in this specification in the context of separate embodiments can also be implemented in combination in a single embodiment in part or in whole. Conversely, various features that are described in the context of a single embodiment can also be implemented in multiple embodiments separately or in any suitable subcombination. Moreover, although features may be described herein as acting in certain combinations and/or initially claimed as such, one or more features from a claimed combination can in some cases be excised from the combination, and the claimed combination may be directed to a subcombination or variation of a subcombination. Similarly, while operations may be described in a particular order, this should not be understood as requiring that such operations be performed in the particular order or in sequential order, or that all operations be performed, to achieve desirable results. Particular embodiments of the subject matter have been described. Other embodiments are within the scope of the following claims.

What is claimed is:

1. A method for classifying data using non-negative matrix factorization, the method comprising:
receiving a population of sample data, wherein the population includes amplicon counts per sample data;
generating a first matrix of the amplicon counts per sample data;
dividing the first matrix into a product of a second matrix and a third matrix, the second matrix being signatures of short and long DNA fragments and the third matrix being intensities of each signature of the short and long DNA fragments;
in the second matrix, determining whether each signature is a long or short fragment per each amplicon count;
in the third matrix, determining intensities of each signature per the sample data; and
classifying the sample data based on the intensities of each signature.

2. The method of claim 1, further comprising normalizing the amplicon counts.

3. The method of claim 1, further comprising filtering the amplicon counts.

4. The method of claim 1, wherein the signatures include a first signature indicative of the short fragment size and a second signature indicative of the long fragment size.

5. The method of claim 4, wherein the short fragment size is indicative of cancer.

6. The method of claim 4, wherein the long fragment size is indicative of normal.

7. The method of claim 4, further comprising assigning a classifier value of 1 to sample data having a greater intensity of the first signature.

8. The method of claim 4, further comprising assigning a classifier value of 0 to sample data having a greater intensity of the second signature.

9. The method of claim 1, further comprising applying a non-negative least square function to the intensities of each signature per each sample data.

10. The method of claim 1, further comprising applying linear regression analysis to the intensities of each signature per each sample data.

11. The method of claim 1, wherein classifying the sample data comprises applying a deep learning model.

12. The method of claim 1, wherein classifying the sample data comprises applying a state vector machine.

13. The method of claim 1, wherein each sample data is a chromosomal arm.

14. The method of claim 1, wherein each sample data is a sequenced DNA sample.

15. The method of claim 1, further comprising iteratively improving one or more algorithms applied in the method.

16. The method of claim 4, wherein the short fragment size is indicative of at least one of adenomatous polyps or advanced adenomas in an organ or tumor.

17. A system for classifying data using non-negative matrix factorization, the system comprising:
one or more processors; and
computer memory storing instructions that, when executed by the processors, cause the processors to perform operations comprising:
receiving a population of sample data, wherein the population includes amplicon counts per sample data;
generating a first matrix of the amplicon counts per sample data;
dividing the first matrix into a product of a second matrix and a third matrix, the second matrix being signatures of short and long DNA fragments and the third matrix being intensities of each signature of the short and long DNA fragments;
in the second matrix, determining whether each signature is a long or short fragment per each amplicon count;
in the third matrix, determining intensities of each signature per the sample data; and
classifying the sample data based on the intensities of each signature.

18. The system of claim 17, wherein the signatures include a first signature indicative of the short fragment size and a second signature indicative of the long fragment size.

19. The system of claim 18, wherein the short fragment size is indicative of cancer.

20. The system of claim 18, wherein the short fragment size is indicative of at least one of an adenomatous polyp or advanced adenoma in an organ or tumor.
